SEQUENCE LISTING (2 sequences)

SEQ ID NO: 1 - protein, 567 aa, Homo sapiens
MWLRAFILATLSASAAWGHPSSPPVVDTVHGKVLGKFVSLEGFAQPVAIFLGIPFAKPPLGPLRFTPPQPAEPWSFVKNATSYPPMCTQDPKAGQLLSELFTNRKENIPLKLSEDCLYLNIYTPADLTKKNRLPVMVWIHGGGLMVGAASTYDGLALAAHENVVVVTIQYRLGIWGFFSTGDEHSRGNWGHLDQVAALRWVQDNIASFGGNPGSVTIFGESAGGESVSVLVLSPLAKNLFHRAISESGVALTSVLVKKGDVKPLAEQIAITAGCKTTTSAVMVHCLRQKTEEELLETTLKMKFLSLDLQGDPRESQPLLGTVIDGMLLLKTPEELQAERNFHTVPYMVGINKQEFGWLIPMQLMSYPLSEGQLDQKTAMSLLWKSYPLVCIAKELIPEATEKYLGGTDDTVKKKDLFLDLIADVMFGVPSVIVARNHRDAGAPTYMYEFQYRPSFSSDMKPKTVIGDHGDELFSVFGAPFLKEGASEEEIRLSKMVMKFWANFARNGNPNGEGLPHWPEYNQKEGYLQIGANTQAAQKLKDKEVAFWTNLFAKKAVEKPPQTEHIEL

SEQ ID NO: 2 - protein, 614 aa, Homo sapiens
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

(19) United States
(12) Patent Application Publication
(10) Pub. No.: US 2012/0309037 A1
(43) Pub. Date: Dec. 6, 2012

(54) Methods and Compositions for Detection and Identification of Organophosphorus Nerve Agents, Pesticides and Other Toxins

(76) Inventors: Matthew Robert Redinbo, Chapel Hill, NC (US); Andrew Clinton Hemmert, Murray, UT (US); Jonathan Seaver Edwards, Boston, MA (US)

(21) Appl. No.: 13/514,744

(22) PCT Filed: Dec. 8, 2010
(86) PCT No.: PCT/US10/59453

§ 371 Date: Aug. 14, 2012

Related U.S. Application Data
(60) Provisional application No. 61/267,591, filed on Dec. 8, 2009.

(51) Int. Cl. C12Q 1/44; G01N 27/00; C12N 11/14; C12Q 1/46; G01N 21/78; G01N 21/75; C12M 1/40; B82Y 15/00

(52) U.S. Cl. 435/20; 435/19; 435/287.1; 435/288.7; 435/176; 977/773; 977/902

(57) ABSTRACT

The present invention provides methods and devices for detecting and identifying toxins, including but not limited to organophosphorus nerve agents and/or organophosphorus pesticides, in a sample. One embodiment of the present invention comprises a method for identifying an organophosphorus nerve agent and/or an organophosphorus pesticide, comprising: exposing a group of enzymes comprising human carboxylesterase 1, at least one mutant of human carboxylesterase 1, and acetylcholinesterase to a sample, wherein the enzymes are separate from each other and each enzyme binds at least one organophosphorus nerve agent or at least one organophosphorus pesticide; contacting the exposed enzymes with a fluid comprising an oxime and a substrate; and detecting a signal produced upon reaction of the substrate and the exposed enzymes, whereby detection of the signal identifies the organophosphorus nerve agent and/or the organophosphorus pesticide.

### RELATED APPLICATION DATA

This application claims the benefit, under 35 U.S.C. §119(e), of U.S. Provisional Patent Application Ser. No. 61/267,591, filed Dec. 8, 2009, the disclosure of which is incorporated herein by reference in its entirety.

### STATEMENT OF GOVERNMENT SUPPORT

Aspects of this invention were supported by funding provided under National Institute of Neurological Disorders and Stroke (NINDS) Grant No. U01 NS58089. The U.S. Government has certain rights to this invention.

### FIELD OF THE INVENTION

The present invention relates to methods and compositions for detection and identification of organophosphorus nerve agents and organophosphorus pesticides.

### BACKGROUND OF THE INVENTION

The organophosphorus (OP) nerve agents tabun (GA), sarin (GB), soman (GD), cyclosarin (GF), VX, and Russian VX (R-VX) are among the deadliest compounds known. Although the military use of OP nerve agents is widely banned, OP nerve agents have been weaponized and used by military forces and civilian terrorists. The level of toxicity of these nerve agents is striking, with LD₅₀ values for percutaneous exposure ranging from 1 g/person for tabun to 10 mg/person for VX. Unfortunately, the current treatments for nerve agent exposure are limited because the current technology does not allow for the nerve agent to be easily identified; only the presence of some nerve agent can be confirmed. Oximes, such as obidoxime, prolidoxime (2-PAM), and HI 6, are often used to treat nerve agent exposure; however, there is no broad spectrum oxime, i.e., there is no oxime that works for all nerve agents. Additionally, an oxime needs to be administered within minutes after exposure; however, oximes are toxic and high doses of oximes may result in respiratory paralysis and hepatoxicity. A further complication is that combining oximes may reduce their effectiveness. Overall, the available treatments for nerve agent exposure offer limited protection, must be administered immediately, and can generate long-term toxic side effects.

Other organophosphorus compounds that can create significant adverse health problems are OP pesticides. Organophosphorus pesticides are widely used, but pose significant health risks upon long-term exposure. OP pesticides also cause environmental pollution. Because of their toxicity and relatively high solubility in water, OP pesticides pose a clear threat to drinking water and aquatic life. It is therefore necessary to monitor the levels of these materials in areas such as industrial waste waters, agricultural runoffs, and other environments to determine compliance with federal and state regulations and other safety guidelines, as well as efficiency of wastewater treatments. Additionally, for clean up or decontamination methods, determining the identity of the OP pesticide aids in choosing the correct method to use for the decontamination process.

Accordingly, there is a need for the development of methods and compositions to detect and identify OP nerve agents and OP pesticides.

Carboxylesterases (CE) are ubiquitous serine esterase enzymes that catalyze the conversion of carboxylic esters to an alcohol and a carboxylic acid and hydrolyze amides, thioesters, phosphoric acid esters and acid anhydrides. CEs recognize a variety of different endogenous and exogenous compounds, including both small and large ligands. In some cases, CE enzyme activity is responsible for the detoxification of xenobiotics. In humans, CEs are present in high levels in both normal and tumor tissue, especially in liver, kidney, testis, lung and plasma. In other species, CEs circulate in the blood and these animals have been shown to be less susceptible to the OP nerve agents and OP pesticides.

Human carboxylesterase 1 (hCE1) is a promiscuous serine hydrolase that binds a broad spectrum of structurally diverse compounds and plays a central role in several key biological processes. This enzyme catalyzes the hydrolysis of esters, thioesters and amide bonds in a wide variety of chemically distinct drugs, xenobiotics and endogenous compounds. Its primary role appears to be in the promiscuous metabolism and detoxification of xenobiotics that pose a potential threat to survival. Due to the natural ability of hCE1 to hydrolyze certain OP nerve agents and its ability to bind structurally diverse compounds it is an ideal candidate for design into an efficient, broad-spectrum OP hydrolase.

Thus, the present invention overcomes previous shortcomings in the art by providing methods and compositions for identifying OP nerve agents and OP pesticides.

### SUMMARY OF THE INVENTION

In one aspect, the present invention provides a method for identifying the presence of a toxin, comprising:

a) exposing a group of enzymes comprising human carboxylesterase 1, at least one mutant of human carboxylesterase 1, and acetylcholinesterase to a sample, wherein the enzymes are separate from each other and each enzyme binds at least one toxin;

b) contacting the exposed enzymes with a fluid comprising an oxime and a substrate; and

c) detecting a signal produced upon reaction of the substrate and the exposed enzymes of step b), whereby detection of the signal identifies the presence of the toxin.

A further aspect of the present invention provides a method for identifying an organophosphorus nerve agent and/or an organophosphorus pesticide, comprising:

a) exposing a group of enzymes comprising human carboxylesterase 1, at least one mutant of human carboxylesterase 1, and acetylcholinesterase to a sample, wherein the enzymes are separate from each other and each enzyme binds at least one organophosphorus nerve agent or at least one organophosphorus pesticide;

b) contacting the exposed enzymes with a fluid comprising an oxime and a substrate; and

c) detecting a signal produced upon reaction of the substrate and the exposed enzymes of step b), whereby detection of the signal identifies the organophosphorus nerve agent and/or the organophosphorus pesticide.

Another aspect of the present invention provides a method for identifying an organophosphorus nerve agent, comprising:

a) exposing a group of enzymes comprising acetylcholinesterase and at least one mutant of human carboxylesterase 1 to a sample, wherein the enzymes are separate from each other and each enzyme binds at least one organophosphorus nerve agent;

b) contacting the exposed enzymes with a fluid comprising an oxime and a substrate; and

c) detecting a signal produced upon reaction of the substrate and the exposed enzymes of step b), whereby detection of the signal identifies the organophosphorus nerve agent.

An additional aspect of the present invention provides a method for identifying an organophosphorus nerve agent, comprising:

a) exposing an enzyme to a sample, wherein the enzyme is a mutant of human carboxylesterase 1 and the enzyme binds at least one organophosphorus nerve agent;

b) contacting the exposed enzyme with a fluid comprising an oxime and a substrate; and

c) detecting a signal produced upon reaction of the substrate and the exposed enzyme of step b), whereby detection of the signal identifies the organophosphorus nerve agent.

A still further aspect of the present invention provides a method for identifying an organophosphorus nerve agent, comprising:

a) exposing an enzyme selected from the group consisting of acetylcholinesterase, V146H/L363Q human carboxylesterase 1, L97K human carboxylesterase 1, V146H/L363E human carboxylesterase 1, and any combination thereof to a sample, wherein each enzyme binds at least one organophosphorus nerve agent selected from the group consisting of sarin, soman, cyclosarin, tabun, VX, VR, and any combination thereof;

b) contacting the exposed enzyme with a fluid comprising an oxime and a substrate; and

c) detecting a signal produced upon reaction of the substrate and the exposed enzyme of step b), whereby detection of the signal identifies the organophosphorus nerve agent.

A further aspect of the present invention provides a method for identifying an organophosphorus nerve agent and/or an organophosphorus pesticide, comprising:

a) exposing an enzyme selected from the group consisting of acetylcholinesterase, human carboxylesterase 1, V146H/L363Q human carboxylesterase 1, L97K human carboxylesterase 1, V146H/L363E human carboxylesterase 1, and any combination thereof to a sample, wherein each enzyme binds at least one organophosphorus nerve agent selected from the group consisting of sarin, soman, cyclosarin, tabun, VX, VR, and any combination thereof or the organophosphorus pesticide paraoxon;

b) contacting the exposed enzyme with a fluid comprising an oxime and a substrate; and

c) detecting a signal produced upon reaction of the substrate and the exposed enzyme of step b), whereby detection of the signal identifies the organophosphorus nerve agent and/or the organophosphorus pesticide.

Additional aspects of the present invention provide a device for identifying at least one toxin (e.g., an organophosphorus nerve agent and/or an organophosphorus pesticide), the device comprising:

a device body comprising a plurality of spaced apart test spots, wherein, in operation, the device body is adapted to be in communication with a sample and allow the sample to communicate with each test spot, wherein each test spot comprises at least one enzyme, and wherein different test spots comprise a different enzyme or a different combination of enzymes from the other test spots; and

a first fluid reservoir holding a first defined fluid comprising a substrate, wherein the first fluid reservoir is attached to the device body, and wherein the first fluid reservoir is configured to controllably release the first defined fluid so that the first defined fluid is in fluid communication with the one or more test spots to expose the first defined fluid to the one or more enzymes, whereby the substrate from the first defined fluid reacts with one or more of the enzymes of a respective test spot if a toxin (e.g., an organophosphorus nerve agent and/or organophosphorus pesticide) is present in the sample to generate a signal that identifies the toxin (e.g., organophosphorus nerve agent and/or organophosphorus pesticide).

Further aspects of the present invention provide a device for identifying at least one toxin (e.g., an organophosphorus nerve agent and/or at organophosphorus pesticide), the device comprising:

a device body comprising a plurality of spaced apart test spots, wherein, in operation, the device body is adapted to be in communication with a sample and allow the sample to communicate with each test spot, wherein each test spot comprises at least one enzyme, and wherein different test spots comprise a different enzyme or a different combination of enzymes from the other test spots;

a first fluid reservoir holding a first defined fluid comprising a substrate, wherein the first fluid reservoir is attached to the device body; and

a second fluid reservoir holding a second defined fluid comprising an oxime, wherein the second fluid reservoir is attached to the first fluid reservoir so that the first defined fluid is separated from the second defined fluid, and wherein the second fluid reservoir is configured to controllably release the second defined fluid into the first defined fluid to produce a combined fluid and the first fluid reservoir is configured to controllably release the combined fluid so that the combined fluid is in fluid communication with the one or more test spots to expose the combined fluid to the one or more enzymes whereby the substrate from the first defined fluid reacts with one or more of the enzymes of a respective test spot if a toxin (e.g., an organophosphorus nerve agent and/or organophosphorus pesticide) is present in the sample to generate a signal that identifies the toxin (e.g., an organophosphorus nerve agent and/or organophosphorus pesticide).

Other aspects of the present invention provide an active encapsulated mutant of human carboxylesterase 1 in a silicate nanoparticle, wherein the mutant is selected from the group consisting of V146H/L363Q human carboxylesterase 1, L97K human carboxylesterase 1, and V146H/L363E human carboxylesterase 1 and any combination thereof.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE FIGURES

FIG. 1 illustrates one embodiment of the device of the present invention.

FIGS. 2A-E show a top view of one embodiment of the device of the present invention and illustrate the signal pattern that would be generated if the OP nerve agents sarin, soman, cyclosarin, tabun, or VX/VR, respectively, are present in a sample.

### DETAILED DESCRIPTION

The present invention will now be described more fully hereinafter with reference to the accompanying drawings and specification, in which various embodiments of the invention are shown. This invention may, however, be embodied in different forms and should not be construed as limited to the embodiments set forth herein. Like numbers refer to like elements throughout. In addition, the sequence of operations (or steps) is not limited to the order presented herein unless specifically indicated otherwise. In the drawings, the thickness of lines, layers, features, components and/or regions may be exaggerated for clarity and broken lines illustrate optional features or operations, unless specified otherwise.

The terminology used herein is for the purpose of describing particular embodiments only and is not intended to be limiting of the invention. As used herein, the singular forms, “a,” “an” and “the” are intended to include the plural forms as well, unless the context clearly indicates otherwise. It will be further understood that the terms “comprises,” “comprising,” “includes,” and/or “including” when used in this specification, specify the presence of stated features, regions, steps, operations, elements, and/or components, but do not preclude the presence or addition of one or more other features, regions, steps, operations, elements, components, and/or groups thereof.

It will be understood that when a feature, such as a layer, region or body, is referred to as being “on” another feature or element, it can be directly on the other feature or element or intervening features and/or elements may also be present. In contrast, when an element is referred to as being “directly on” another feature or element, there are no intervening elements present. It will also be understood that, when a feature or element is referred to as being “connected,” “attached” or “coupled” to another feature or element, it can be directly connected, attached or coupled to the other element or intervening elements may be present. In contrast, when a feature or element is referred to as being “directly connected,” “directly attached” or “directly coupled” to another element, there are no intervening elements present. Although described or shown with respect to one embodiment, the features so described or shown can apply to other embodiments.

It will be understood that although the terms “first,” “second,” and “third,” etc., are used herein to describe various components, regions, layers and/or sections, these regions, layers and/or sections should not be limited by these terms. These terms are only used to distinguish one component, region, layer or section from another component, region, layer or section. Thus, a first component, region, layer or section discussed below could be termed a second component, region, layer or section, and vice versa, without departing from the teachings of the present invention. Like numbers refer to like elements throughout.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

The term “about,” as used herein when referring to a measurable value such as an amount or concentration (e.g., an amount of a nerve agent) and the like, is meant to encompass variations off 20%, ±10%, ±5%, ±1%, ±0.5%, or even ±0.1% of the specified amount.

Embodiments of the present invention are directed to methods and compositions for the detection and/or identification of toxins. “Toxin” as used herein refers to a chemical or biological compound or mixture of such compounds that is poisonous and/or damaging to living cells, organisms, and/or the environment. Exemplary toxins include, but are not limited to, nerve agents, pesticides, insecticides, herbicides, solvents, and plasticizers. The toxin may contain esters, thioesters, phosphoric acid esters, acid anhydrides, or amide bonds. In some embodiments of the present invention, the toxin is an organophosphorus compound. The toxin can, in some embodiments, be hydrolyzed by an enzyme, such as a serine hydrolase or a mutant of a serine hydrolase. In some embodiments, the toxin of this invention can be an organophosphorous toxin.

As used herein, the term “nerve agent” refers to a chemical compound that disrupts the functioning of the nervous system of an organism, such as by inhibiting the actions of the enzyme acetylcholinesterase. The nerve agent can be any organophosphorus nerve agent, including, but not limited to G-type nerve agents and V-type nerve agents. Exemplary organophosphorus nerve agents include: tabun (GA), sarin (GB), soman (GD), cyclosarin (GF), VX, Russian VX (R-VX), VR, VE, VG, and VM.

As used herein, the term “pesticide” refers to a chemical or biological substance that deters, prevents, kills, damages, destroys, or repels any pest, such as insects, plant pathogens, weeds, molluscs, birds, mammals, fish, nematodes, and microbes. The term “pesticide” includes, but is not limited to, insecticides, herbicides, fungicides, rodenticides, and biocides. The pesticide can contain an ester, thioester, phosphoric acid ester, acid anhydride, or amide bond. In some embodiments, the pesticide is an organophosphorus pesticide, such as but not limited to chlorpyrifos, dichlorvos, malathion, methyl parathion, parathion, diazinon, paraoxon, chlorpyrifos oxon, malaoxon, methyl paraoxon, and diazinon oxon.

“Insecticide” as used herein refers to a pesticide that is used to deter, prevent, kill, damage, destroy, or repel an insect. In some embodiments of the present invention, the insecticide is an organophosphorus insecticide. Exemplary insecticides include disulfoton, phorate, dimethoate, ciodrin, dichlorvos, dioxathion, ruelene, carbophenothion, supona, TEPP, EPN, HETP, parathion, malathion, ronnel, coumaphos, diazinon, trichlorfon, paraoxon, potasan, dimefox, mipafox, schradan, sevin, chlorpyrifos, dimeton, chlorthion, and fenchlorphos.

“Herbicide” as used herein refers to a pesticide that is used to deter, prevent, kill, damage, destroy, or repel a weed or unwanted plant. In some embodiments of the present invention, the herbicide is an organophosphorus herbicide. Exemplary herbicides include amiprofos-methyl, amiprophos, anilofos, bensulide, bilanafos, butamifos, 2,4-DEP, DMPA, EBEP, fosamine, glufosinate, glufosinate-P, glyphosate, huangcaoling, piperophos, dimethfuron, triclopyr, atrazine, and dicamba.

“Plasticizer” as used herein refers to a chemical substance that increases the fluidity or plasticity of a material to which it is added. Exemplary plasticizers include tris-2-chhloroisopropyl phosphate (TCPP), triphenyl phosphate (TPP), tributyl phosphate (TBP), and tris(2-butoxyethyl) phosphate (TBEP).

Without being limited to a particular toxin, exemplary embodiments of the present invention are described herein in which the toxin is a nerve agent and/or pesticide.

As used herein, the term “detecting” means confirming or establishing the presence of a toxin (e.g., a nerve agent and/or pesticide) by determining or identifying the presence of a signal that appears and/or is produced in the presence of the toxin (e.g., nerve agent and/or pesticide).

As used herein, the term “identifying” means specifically determining or verifying that a detected toxin (e.g., nerve agent and/or pesticide) is a particular toxin (e.g., nerve agent and/or pesticide).

Thus, the present invention is based on the discovery that toxins (e.g., nerve agents and/or pesticides) can be rapidly detected and identified in a sample, allowing for immediate treatment and/or decontamination employing the appropriate agents.

Thus, one embodiment of the present invention comprises a method for identifying a toxin (e.g., an organophosphorus nerve agent and/or an organophosphorus pesticide), comprising:

a) exposing a group of enzymes comprising human carboxylesterase 1, at least one mutant of human carboxylesterase 1, and acetylcholinesterase to a sample, wherein the enzymes are separate from each other and each enzyme binds at least one toxin (e.g., organophosphorus nerve agent and/or organophosphorus pesticide;

b) contacting the exposed enzymes with a fluid comprising an oxime and a substrate; and

c) detecting a signal produced upon reaction of the substrate and the exposed enzymes of step b), whereby detection of the signal identifies the toxin (e.g., the organophosphorus nerve agent and/or the organophosphorus pesticide).

The method of the present invention can be performed by any method known to one skilled in the art. Non-limiting examples include performing the method in a reaction vessel, such as in a microplate, beaker, etc., or on or in a device. Exemplary ways that the enzyme can be exposed to the sample include: directly adding or applying the sample to the enzyme or vice versa, contacting the enzyme to the sample or vice versa, flowing the sample over the enzyme, and/or exposing or placing the enzyme in an environment to allow the sample to come into contact with the enzyme.

As used herein, the term “enzyme” refers to a serine hydrolase or a mutant of a serine hydrolase from any animal, such as a mammal. In one embodiment the enzyme is from a human, but in other embodiments the enzyme may be from a non-human mammal, such as a mouse, rat, dog, cow, rabbit, or guinea pig. Exemplary serine hydrolases include but are not limited to carboxylesterases and cholinesterases, such as acetylcholinesterases and butyrylcholinesterases as well as mutants of these serine hydrolases. In some embodiments the enzyme or group of enzymes can be human carboxylesterase 1 (e.g., GenBank® Database Accession Number AAA35649.1 (incorporated by reference herein) (MWLRAFILATLSASAAWGHPSSPPVVDTVHGKVLGKFVSLEGFAQPVAIFLGIPFAK PPLGPLRFTPPQPAEPWSFVKNATSYPPMCTQDPKCAGQLLSELFTNRKENIPLKLSED CLYLNIYTPADLTKKNRLPVMVWIHGGGLMVGAASTYDGLALAAHENVVVVTIQY RLGIWGFFSTGDEHSRGNWGHLDQVAALRWVQDNIASFGGNPGSVTIFGESAGGES VSVLVLSPLAKNLFHRAISESGVALTSVLVKKGDVKPLAEQIAITAGCKTTTSAVMV HCLRQKTEEELLETTLKMKFLSLDLQGDPRESQPLLGTVIDGMLLLKTPEELQAERNF HTVPYMVGINKQEFGWLIPMQLMSYPLSEGQLDQKTAMSLLWKSYPLVCIAKELIPE ATEKYLGGTDDTVKKKDLFLDLIADVMFGVPSVIVARNHRDAGAPTYMYEFQYRPS FSSDMICPKTVIGDHGDELFSVFGAPFLICEGASEEEIRLSKMVMKFWANFARNGNPN GEGLPHWPEYNQKEGYLQIGANTQAAQICLICDKEVAFWTNLFAKICAVEKPP QTEHIEL; SEQ ID NO. 1)), mutants of human carboxylesterase 1, acetylcholinesterase, (e.g., GenBank® Database Accession Number AAA68151.1 (incorporated by reference herein) (MRPPQCLLHT PSLASPLLLL LLWLLGGGVG AEGREDAELL VTVRGGRLRG IRLKTPGGPVSAFLGIPFAE PPMGPRRFLP PEPKQPWSGV VDATTFQSVC YQYVDTLYPG FEGTEMWNPNRELSEDCLYL NVWTPYPRPT SPTPVLVWIY GGGFYSGASS LDVYDGRFLV QAERTVLVSMNYRVGAFGFL ALPGSREAPG NVGLLDQRLA LQWVQENVAA FGGDPTSVTL FGESAGAASVGMHLLSPPSR GLFHRAVLQS GAPNGPWATV GMGEARRRAT QLAHLVGCPP GGTGGNDTEL VACLRTRPAQ VLVNHEWHVL PQESVFRFSF VPVVDGDFLS DTPEALINAG DFHGLQVLVGVVKDEGSYFL VYGAPGFSKD NESLISRAEF LAGVRVGVPQ VSDLAAEAVV LHYTDWLHPE DPARLREALS DVVGDHNVVC PVAQLAGRLA AQGARVYAYV FEHRASTLSW PLWMGVPHGY EIEFIFGIPL DPSRNYTAEE KIFAQRLMRY WANFARTGDP NEPRDPKAPQ WPPYTAGAQQ YVSLDLRPLE VRRGLRAQAC AFWNRFLPKL LSATDTLDEA ERQWKAEFHR WSSYMVHWKN QFDHYSKQDR CSDL; SEQ ID NO:2)) and/or mutants of acetylcholinesterase. The enzymes of the present invention may comprise a specific individual enzyme or a combination of different enzymes that are exposed to a sample or samples. In one embodiment of the present invention multiple enzymes are exposed to a sample or samples, but each enzyme is individually contained or separated from the other enzymes.

As used herein, the term “mutant” refers to an enzyme, such as a carboxylesterase or acetylcholinesterase, which comprises, consists essentially of or consists of at least one amino acid substitution, i.e., the mutant can comprise 1, 2, 3, 4, 5, 6, 7, 8, 9, or 10 amino acid substitutions. The term “mutant” as used herein also refers to an enzyme that comprises, consists essentially of or consists of an insertion or deletion of amino acids, wherein the amino acids inserted or deleted may be 0, 1, 2, 3, 4, 5, 6, 7, 8, 9, or 10. Additionally, the term “mutant” as used herein may comprise, consist essentially of or consist of a mutation caused by any combination of a substitution, insertion, or deletion of amino acids.

Non-limiting examples of mutants of human carboxylesterase 1 include V146H/L363Q human carboxylesterase 1, L97K human carboxylesterase 1, V146H/L363E human carboxylesterase 1, L97H human carboxylesterase 1, V146H/L97H human carboxylesterase 1, L363K human carboxylesterase 1, V146H/A93E human carboxylesterase 1, V146H/A93E/1421F human carboxylesterase 1, L363E human carboxylesterase 1, and any combination thereof. In one embodiment of the present invention V146H/L363Q human carboxylesterase 1, L97K human carboxylesterase 1, and V146H/L363E human carboxylesterase 1 are used to identify OP nerve agents by exposing each separately contained enzyme to a sample. Numbering of the amino acids of the mutants is based on the amino acid sequence of human carboxylesterase 1, as set forth in GenBank® Database Accession Number AAA35649.1 (SEQ ID NO:1).

Additionally, the phenotype of the mutants of the present invention encompasses enzymes that have a beneficial impact on nerve agent hydrolysis. This phenotype includes enzymes that reactivate with at least one OP nerve agent and/or OP pesticide different from the wild-type enzyme. The phenotype also includes enzymes that hydrolyze at least one OP nerve agent and/or OP pesticide with a reduced hydrolysis half-time and/or a reduced reactivation half-time relative to the wild-type enzyme or other enzymes that bind the OP nerve agent or OP pesticide. For instance, hCE1 hydrolyzes the OP nerve agent sarin with a half-time of reactivation of approximately 47 hours and this half-time of reactivation can be reduced to 45 minutes with the addition of diacetyl monoxime (DAM). However, hCE1 exhibits no reactivation with other G-type agents such as soman, cyclosarin, and tabun. The double mutant V146H/L363Q human carboxylesterase 1 hydrolyzes sarin with a half-time of 2.9 hours and the addition of DAM reduces the reactivation half-time to approximately 10 minutes. The mutant L97K human carboxylesterase 1 hydrolyzes soman with a half-time of about 1 hour about 20 times faster than wild-type hCE1. The double mutant V146H/L363E human carboxylesterase 1 hydrolyzes cyclosarin with a half time of about 1.2 hours and has a reactivation half-time that is greater than about 1,000 times faster than wild-type hCE1. Thus, these mutants of hCE1 reactivate a different OP nerve agent than wild-type hCE1 and exhibit a reactivation rate that is faster than wild-type hCE1. In some embodiments of the present invention the reactivation rate of the mutant enzymes is about 2, 4, 8, 10, 15, 20, 50, or 100 times faster than the wild type enzyme. The half-time of reactivation is determined by dividing the natural log of 2 by the rate of reactivation (Millard et al., 1998).

The enzymes of the present invention may be present in a fluid, liquid, aqueous solution, gel, and/or solid. Additionally, the enzymes of the present invention may be unconjugated or conjugated with other enzymes, proteins, and/or compounds. The enzymes of the present invention may also be embedded or immobilized on a surface or solid. In one embodiment of the present invention, the enzymes can be encapsulated in a silicate nanoparticle. In another embodiment of the present invention, the enzymes can be PEGylated. In a further embodiment of the present invention, the enzymes can be biotinylated. In still another embodiment of the present invention, the enzymes can be unconjugated in a fluid.

The enzymes of the present invention each detect at least one OP nerve agent and/or OP pesticide. For example, V146H/L363Q human carboxylesterase 1 can detect sarin, VX, and VR; L97K human carboxylesterase 1 can detect soman, VX, and VR; V146H/L363E human carboxylesterase 1 can detect cyclosarin, VX, and VR; L97H human carboxylesterase 1, V146H/L97H human carboxylesterase 1, L363K human carboxylesterase 1, V146H/A93E human carboxylesterase 1, and V146H/A93E/1421F human carboxylesterase 1 can detect sarin; L363E human carboxylesterase 1 can detect soman and cyclosarin; acetylcholinesterase can detect tabun; and human carboxylesterase 1 can detect paraoxon.

As used herein, the term “sample” refers to any gaseous, fluid, liquid, aqueous, gel, aerosol, or solid sample in which a nerve agent and/or pesticide can be detected. A sample can be from any source and can include, but is not limited to, a physiological, environmental, biological, chemical, agricultural and/or industrial source. The sample may or may not be collected for testing. For instance, the sample may comprise or consist of open air testing or continuous testing (e.g., of a fluid, gas, solid, etc.). Alternatively, the sample could be a surface, environment, article, and/or area known or suspected to contain and/or be contaminated with a nerve agent and/or pesticide and/or could be a fluid, air, food, sewage, or soil sample collected for testing. Additionally, the sample could change as the environment changes, e.g., the sample could be a gaseous sample when testing a given area or environment and later change to a liquid or aerosol sample when a liquid or aerosol is present in the area or environment.

In one embodiment, after the enzymes are exposed to the sample, the exposed enzymes are contacted with a fluid comprising, consisting essentially of, or consisting of an oxime and a substrate. The fluid may be a liquid, gas, aerosol, or aqueous solution. The fluid can be contacted with the exposed enzymes by any means known in the art, such as but not limited to, flowing the fluid over the exposed enzymes, adding the fluid to the exposed enzymes or vice versa, mixing the fluid with the exposed enzymes, etc.

As used herein, the term “oxime” refers to any oxime that is able to reactivate the enzyme, i.e., that is able to release the bound organophosphorus nerve agent, pesticide, substrate, or any combination thereof from the enzyme. Exemplary oximes include but are not limited to diacetyl monoxime, monoisonitrosoacetone, obidoxime, and any combination thereof. The fluid may comprise, consist essentially of, or consist of a combination of 0, 1, 2, 3, 4, 5 or more oximes. In one embodiment the oxime is diacetyl monoxime or obidoxime. In another embodiment the oxime is diacetyl monoxime and obidoxime.

As used herein, the term “substrate” refers to any compound or reagent that binds to or reacts with the enzyme, nerve agent, and/or pesticide to produce a signal that allows for the detection and identification of the nerve agent and/or pesticide. In one embodiment of the present invention the exposed enzymes are contacted with the fluid under conditions whereby the exposed enzymes can react with the substrate in the fluid to allow for detection and identification of an organophosphorus nerve agent and/or pesticide. Exemplary substrates include radiological substrates, spectroscopic substrates, colorimetric substrates and substrates that cause or produce an electrical change in the environment. Colorimetric substrates include those substrates that are optically detectable, such as by color, chemiluminescence, or fluorescence; however, colorimetric substrates are not limited to color changes or the production of colored substances.

As used herein, the term “optically detectable” refers to the detection of a signal that can be seen with the naked human eye or visually with the aid of any device, e.g., microscope, array detector, camera, video recording device, etc. Colorimetric substrates include substrates that can create some optically detectable change, such as in physical appearance (e.g., gas bubbles, etc.) to signal the detection and identification of a nerve agent and/or pesticide. Substrates that cause an electrical change are electrically detectable and include those substrates that cause e.g., a pH change.

As used herein, the term “electrically detectable” refers to the detection of an electrical signal, e.g., a voltage or pH change. Accordingly the term “signal” as used herein depends upon the type of substrate used and how the substrate detects and identifies the nerve agent and/or pesticide, as one skilled in the art would recognize. For instance, in one embodiment the substrate is p-nitrophenyl butyrate, which provides a signal that can be optically detected by the production of a colored substance and/or electronically detected by a change in pH. In another embodiment of the invention the substrate is o-nitrophenyl acetate, which provides a signal that can be optically detected by the production of a colored substance and/or electronically detected by a change in pH.

The fluid may comprise, consist essentially of, or consist of a pH adjusting agent and/or buffer, as would be well known in the art. Exemplary pH adjusting agents and buffers include, but are not limited to, acids such as acetic, boric, citric, lactic, phosphoric and hydrochloric acids; bases such as sodium hydroxide, sodium phosphate, sodium borate, sodium citrate, sodium acetate, potassium phosphate and tris-hydroxymethylaminomethane, triethanolamine; and buffers such as potassium phosphate, citrate/dextrose, sodium bicarbonate, ammonium chloride, 3-{[tris(hydroxymethyl)methyl]amino}propanesulfonic acid, tris(hydroxymethyl)methylamine, N-tris(hydroxymethyl)methylglycine, 4-2-hydroxyethyl-1-piperazincethanesulfonic acid, and 3-(N-morpholino)propanesulfonic acid. In some embodiments, the pH of the compositions is about 5 to about 9, about 6.5 to about 8, about 6.8 to about 7.5, or about 7 to about 7.4. In some embodiments the pH is 7.0, 7.1, 7.2, 7.3, 7.4, 7.5, 7.6, 7.8, 7.9, or 8.0.

Once the fluid is contacted with the exposed enzymes if a specific OP nerve agent and/or OP pesticide is present in the sample, then a signal is produced upon reaction of the substrate in the fluid with the exposed enzyme(s). Detection of the signal establishes the presence of an OP nerve agent and/or pesticide and identifies the particular OP nerve agent and/or particular OP pesticide. In some embodiments the identification of the nerve agent and/or pesticide is established by observing the signal pattern (FIGS. 2A-E), such as by observing or determining the presence or absence of a signal after different enzymes have been exposed to the sample.

The substrate used in the method or device determines the type of signal produced. In one embodiment of the present invention the signal is optically detectable, such as by a color change, the production of a colored substance, or some other change in the physical appearance of the mixture. In another embodiment of the present invention the signal is electronically detectable, such as by a pH change or voltage change. In some embodiments of the present invention the detection of the signal is passive. As used herein, the term “passive” refers to a system or device that requires no external or internal power source. Thus, the signal is detectable without utilizing an internal or external power source.

In another embodiment of the present invention, the method further comprises rinsing the exposed enzyme(s) with a second fluid comprising, consisting essentially of, or consisting of an oxime. The oxime releases any bound nerve agent, pesticide, and/or substrate from the exposed enzyme(s) and allows for the enzyme(s) to be used again for subsequent testing.

In some embodiments of the present invention, the OP nerve agent and/or OP pesticide has a concentration from about 1 μM to about 100 mM, from about 1 μM to about 10 mM, from about 10 μM to about 1 mM, or from about 1, 2, 3, 4, 5, 6, 7, 8, 9, 10, 20, 30, 40, 50, 60 70, 80, 90, 100 μM or any number in between.

In some embodiments of the present invention the identification of the OP nerve agent and/or OP pesticide is accomplished in about 30 seconds to about 1 hour, in about 30 seconds to about 30 minutes, in about 30 seconds to about 10 minutes, in about 30 seconds to about 5 minutes, in about 30 seconds to about 3 minutes, or in about 30, 45, or 60 seconds, or in about 1, 2, 3, 4, 5, 6, 7, 8, 9, or 10 minutes.

In another embodiment of the present invention, the method comprises a method for identifying an organophosphorus nerve agent, comprising:

a) exposing a group of enzymes comprising acetylcholinesterase and at least one mutant of human carboxylesterase 1 to a sample, wherein the enzymes are separate from each other and each enzyme binds at least one organophosphorus nerve agent;

b) contacting the exposed enzymes with a fluid comprising an oxime and a substrate; and

c) detecting a signal produced upon reaction of the substrate and the exposed enzymes of step b), whereby detection of the signal identifies the organophosphorus nerve agent.

In another embodiment of the present invention, the method comprises a method for identifying an organophosphorus nerve agent, comprising:

a) exposing an enzyme to a sample, wherein the enzyme is a mutant of human carboxylesterase 1 and the enzyme binds at least one organophosphorus nerve agent;

b) contacting the exposed enzyme with a fluid comprising an oxime and a substrate; and

c) detecting a signal produced upon reaction of the substrate and the exposed enzyme of step b), whereby detection of the signal identifies the organophosphorus nerve agent.

In another embodiment of the present invention the method comprises a method for identifying an organophosphorus nerve agent and/or an organophosphorus pesticide, comprising:

a) exposing an enzyme selected from the group consisting of acetylcholinesterase, human carboxylesterase 1, V146H/L363Q human carboxylesterase 1, L97K human carboxylesterase 1, V146H/L363E human carboxylesterase 1, and any combination thereof to a sample, wherein each enzyme binds at least one organophosphorus nerve agent selected from the group consisting of sarin, soman, cyclosarin, tabun, VX, VR, and any combination thereof or at least one organophosphorus pesticide selected from the group consisting of paraoxon, methyl parathion, parathion, diazinon, and any combination thereof;

b) contacting the exposed enzyme with a fluid comprising an oxime and a substrate; and

c) detecting a signal produced upon reaction of the substrate and the exposed enzyme of step b), whereby detection of the signal identifies the organophosphorus nerve agent and/or the organophosphorus pesticide.

In another embodiment of the present invention the method comprises a method for identifying an organophosphorus nerve agent, comprising:

a) exposing an enzyme selected from the group consisting of acetylcholinesterase, V146H/L363Q human carboxylesterase 1, L97K human carboxylesterase 1, V146H/L363E human carboxylesterase 1, and any combination thereof to a sample, wherein each enzyme binds at least one organophosphorus nerve agent selected from the group consisting of sarin, soman, cyclosarin, tabun, VX, VR, and any combination thereof;

b) contacting the exposed enzyme with a fluid comprising an oxime and a substrate; and

c) detecting a signal produced upon reaction of the substrate and the exposed enzyme of step b), whereby detection of the signal identifies the organophosphorus nerve agent.

A further embodiment of the present invention comprises a device for identifying at least one organophosphorus nerve agent and/or at least one organophosphorus pesticide, the device comprising:

a device body comprising a plurality of spaced apart test spots, wherein, in operation, the device body is adapted to be in communication with a sample and allow the sample to communicate with each test spot, wherein each test spot comprises at least one enzyme, and wherein different test spots comprise a different enzyme or a different combination of enzymes from the other test spots; and

a first fluid reservoir holding a first defined fluid comprising a substrate, wherein the first fluid reservoir is attached to the device body, and wherein the first fluid reservoir is configured to controllably release the first defined fluid so that the first defined fluid is in fluid communication with the one or more test spots to expose the first defined fluid to the one or more enzymes, whereby the substrate from the first defined fluid reacts with one or more of the enzymes of a respective test spot if an organophosphorus nerve agent and/or organophosphorus pesticide is present in the sample to generate a signal that identifies the organophosphorus nerve agent and/or organophosphorus pesticide.

As used herein, the term “device body” refers to any member that comprises a plurality of test spots. The body may be conformable, flexible, or rigid or have portions that are conformable, flexible, and/or rigid. The device body may be any size or shape. In some embodiments the device body is portable in a configured size and shape and in further embodiments the device body is configured to be a hand held device. Non-limiting examples of a device body include a membrane, film, sponge, patch, housing, or container. The device body may comprise one layer, two layers, or even more layers that can be stacked, laminated, or otherwise attached. The device body may comprise one or more components, layers or materials. If the device body comprises multiple layers, materials, or components, then the different layers, materials or components may comprise the same materials or different materials. In some embodiments the device body is a sponge. In another embodiment the device body is a microplate comprising one or more wells or a plurality of wells. In other embodiments the device body is a patch or badge that can be worn. In some embodiments the device body can be adhesively applied to a surface, e.g., wall, clothing, table, etc.

As used herein, the term “test spots” refers to locations on the device body that comprise, consist essentially of, or consist of at least one enzyme. The test spots may be located anywhere on the device body including, but not limited to, the surface, in or between one or more layers or components of the device body, and/or in the device body. The test spots may be open to the environment or they may be enclosed in the device body. The test spots are not necessarily circular; they can be any shape such as circular, rectangular, etc. In some embodiments the test spots are located on the surface of the device body and are open to the environment. In other embodiments the test spots are located in the device body. In further embodiments the test spots are in a housing or container. There may be one or more test spots on the device body. In some embodiments there are 1, 2, 3, 4, 5, 6, 7, 8, 9, 10, 11, 12, 13, 14, 15, 16, 17, 18, 19, or 20 test spots on the device body. The test spots may be arranged on the device body in any manner, such as a pattern or randomly arranged. In some embodiments the test spots are spaced apart from one another. In some embodiments different test spots can comprise, consist essentially of or consist of a different enzyme or a different combination of enzymes, e.g., there may be more than one test spot containing the same enzyme or the same combination of enzymes but different test spots comprise different enzymes or different combinations of enzymes.

As used herein, the term “fluid reservoir” refers to any housing or container. In some embodiments the fluid reservoir holds a “defined fluid.” As used herein, the term “defined fluid” refers to any liquid, gas, aerosol, or aqueous solution. The fluid reservoir can be open to the environment such that the defined fluid is contained but exposed to the environment or the fluid reservoir can entirely enclose the defined fluid. The fluid reservoir may comprise one or more openings or be configured to allow for access to the fluid reservoir. The fluid reservoir can be attached to the device body, another fluid reservoir, a reservoir barrier, and/or any combination thereof. In some embodiments the fluid reservoir is not removable from the device. In other embodiments the fluid reservoir is attachable and detachable from the device.

The fluid reservoir can be configured to controllably release a defined fluid by any means known in the art, such as, but not limited to, opening, sliding, removing, or puncturing the floor, ceiling or side of the fluid reservoir. The fluid reservoir can be configured to have a rigid or flexible floor, ceiling and/or sides, which confine the defined fluid until the desired time of release to a desired location. In some embodiments the fluid reservoir is configured to controllably release a defined fluid by opening the fluid reservoir. In other embodiments the fluid reservoir does not release the defined fluid, instead a reservoir barrier releases the defined fluid.

As used herein, the term “reservoir barrier” refers to any structure that separates the fluid reservoir from another fluid reservoir and/or the test spots and contains the defined fluid until the time the defined fluid is to be released. The reservoir barrier is configured to allow for the defined fluid to be controllably released to a desired location at the desired point in time. The reservoir barrier can be a solid, semi-solid, rigid, or flexible structure that is opened, removed, punctured or broken at the desired point in time. In one embodiment the reservoir barrier is a rigid structure that is opened or removed. In another embodiment the reservoir barrier is a membrane that can be broken, pierced or punctured at the desired point in time.

In one embodiment of the present invention the device comprises a first fluid reservoir holding a first defined fluid comprising a substrate, wherein the first fluid reservoir is attached to the device body, and wherein the first fluid reservoir is configured to controllably release the first defined fluid so that the first defined fluid is in fluid communication with the one or more test spots to expose the first defined fluid to the one or more enzymes, whereby the substrate from the first defined fluid reacts with one or more of the enzymes of a respective test spot if an organophosphorus nerve agent and/or organophosphorus pesticide is present in the sample to generate a signal that identifies the organophosphorus nerve agent and/or organophosphorus pesticide. In another embodiment the first defined fluid further comprises an oxime.

In a still further embodiment the device further comprises a first reservoir barrier that resides between the first fluid reservoir and the test spots and is configured to controllably release the first defined fluid. In some embodiments only the first reservoir barrier or the first fluid reservoir are configured to controllably release the first defined fluid, but in other embodiments both the first reservoir barrier and the first fluid reservoir are configured to controllably release the first defined fluid to be in fluid communication with the one or more test spots.

In some embodiments the device further comprises a second fluid reservoir holding a second defined fluid comprising an oxime, wherein the second fluid reservoir is attached to the first fluid reservoir and the second fluid reservoir is configured to controllably release the second defined fluid. In other embodiments a second reservoir barrier or both the second reservoir barrier and the second fluid reservoir are configured to controllably release the second defined fluid. The second defined fluid may be released into the first defined fluid or vice versa to produce a combined fluid or the second defined fluid may be released to be in fluid communication with the one or more test spots. In one embodiment the first defined fluid comprises a substrate and an oxime and the second defined fluid comprises an oxime; in this embodiment the second defined fluid is released to be in fluid communication with the one or more test spots after the one or more enzymes have had time to react with the substrate in the first defined fluid to generate a signal. In this embodiment the oxime will release the substrate, OP nerve agent, and/or OP pesticide from the enzyme. Thus, the second defined fluid will wash the one or more enzymes and allow for the one or more enzymes to be reused for subsequent testing. In another embodiment the first defined fluid comprises a substrate and the second defined fluid comprises an oxime; in this embodiment the second defined fluid is combined with the first defined fluid to produce a combined fluid. In some embodiments it is the first reservoir barrier and/or the first fluid reservoir that releases the first defined fluid or the combined fluid, but in other embodiments it is the second reservoir barrier and/or the second fluid reservoir that releases the second defined fluid or the combined fluid.

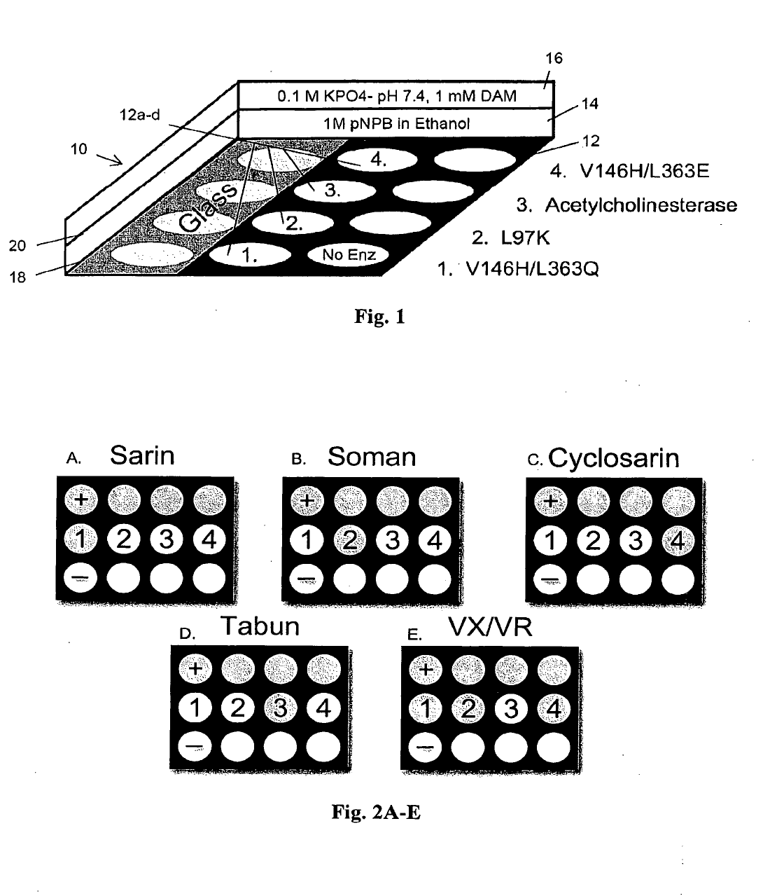

One embodiment of the present invention, as exemplified in FIG. 1, provides a device 10 for identifying at least one organophosphorus nerve agent and/or at least one organophosphorus pesticide, the device comprising:

a device body 12 comprising a plurality of spaced apart test spots 12a-d, wherein, in operation, the device body 12 is adapted to be in communication with a sample and allow the sample to communicate with each test spot, wherein each test spot comprises at least one enzyme, and wherein different test spots comprise a different enzyme or a different combination of enzymes from the other test spots;

a first fluid reservoir 14 holding a first defined fluid comprising a substrate, wherein the first fluid reservoir is attached to the device body; and

a second fluid reservoir 16 holding a second defined fluid comprising an oxime, wherein the second fluid reservoir 16 is attached to the first fluid reservoir 14 so that the first defined fluid is separated from the second defined fluid, and wherein the second fluid reservoir 16 is configured to controllably release the second defined fluid into the first defined fluid to produce a combined fluid and the first fluid reservoir 14 is configured to controllably release the combined fluid so that the combined fluid is in fluid communication with the one or more test spots 12a-d to expose the combined fluid to the one or more enzymes whereby the substrate from the first defined fluid reacts with one or more of the enzymes of a respective test spot if an organophosphorus nerve agent and/or organophosphorus pesticide is present in the sample to generate a signal that identifies the organophosphorus nerve agent and/or organophosphorus pesticide.

In this embodiment the first fluid reservoir and the second fluid reservoir may be arranged so that they are beside each other, one may be on top of the other, and/or they may be located or positioned apart from each other. The first fluid reservoir and the second fluid reservoir may be the same size or one may be larger than the other. In some embodiments the first fluid reservoir and/or the second fluid reservoir can be separate from or unattached to the device. In other embodiments the first fluid reservoir and/or the second fluid reservoir are part of the device and configured to be attached and removed from the device.

In some embodiments the second fluid reservoir is configured to controllably release the second defined fluid into the first defined fluid or vice versa to produce a combined fluid, and the first fluid reservoir is configured to controllably release the combined fluid so that the combined fluid is in fluid communication with the one or more test spots. However, depending on how the first fluid reservoir and the second fluid reservoirs are arranged, the first fluid reservoir may be configured to controllably release the first defined fluid into the second defined fluid or vice versa, and the second fluid reservoir may be configured to controllably release the combined fluid to the one or more test spots. Additionally, in some embodiments a first and/or second reservoir barrier is present and is configured to controllably release either the first defined fluid, the second defined fluid, or the combined fluid.

In one embodiment the device comprises a first fluid reservoir, a second fluid reservoir, and a first reservoir barrier 18 that resides between the first fluid reservoir or the second fluid reservoir and the test spots and is configured to controllably release the combined fluid. The first fluid reservoir, the second fluid reservoir and/or the first reservoir barrier can be configured to controllably release the combined fluid following contact of the one or more enzymes with the sample. After release of the combined fluid, the combined fluid is in fluid communication with the one or more test spots. The combined fluid contacts the one or more test spots and the one or more enzymes are exposed to the combined fluid. The one or more enzymes can then react with the substrate in the combined fluid to generate a signal that is used to identify the organophosphorus nerve agent and/or organophosphorus pesticide present in the sample.

An additional embodiment of the device comprises a second reservoir barrier 20 that resides between the first fluid reservoir and the second fluid reservoir. The second reservoir barrier is configured to controllably release the second defined fluid into the first defined fluid or vice versa to produce a combined fluid.

The first defined fluid and the second defined fluid may be the same or different from each other. In one embodiment, the first defined fluid comprises, consists essentially of, or consists of a substrate and the second defined fluid comprises, consists essentially of, or consists of an oxime. When the device is in operation the second fluid reservoir and/or the second reservoir barrier are configured to controllably combine the first defined fluid and the second defined fluid to produce a combined fluid. The combined fluid may be produced before, during, or after the enzymes are in communication with the sample as long as the first defined fluid and the second defined fluid are combined to produce a combined fluid before they are controllably released by the first fluid reservoir and/or first reservoir barrier to contact the one or more test spots.

In the devices of the present invention the device body comprises a plurality of test spots and each test spot comprises at least one enzyme. In one embodiment the enzymes are selected from the group consisting of acetylcholinesterase, mutant acetylcholinesterases, human carboxycholinesterase, mutant carboxylesterases, and any combination thereof. In another embodiment of the present invention each test spot comprises a single enzyme, the enzymes comprising: V146H/L363Q human carboxylesterase 1, L97K human carboxylesterase 1, V146H/L363E human carboxylesterase 1, and acetylcholinesterase. In this embodiment if sarin is present, then a signal will be generated in the test spot comprising V146H/L363Q human carboxylesterase 1; if soman is present, then a signal will be generated in the test spot comprising L97K human carboxylesterase 1; if cyclosarin is present, then a signal will be generated in the test spot comprising V146H/L363E human carboxylesterase 1; if tabun is present, then a signal will be generated in the test spot comprising acetylcholinesterase; if VX and/or VR are present, then a signal will be generated in the test spots comprising V146H/L363Q human carboxylesterase 1, L97K human carboxylesterase 1, and V146H/L363E human carboxylesterase 1. Thus, if a specific nerve agent is present in the sample, then the identity of each of these nerve agents can be determined by identifying the signal pattern present on the device body (FIGS. 2A-E).

In another embodiment of the present invention the device body comprises a positive control and/or a negative control for one or more of the test spots. In some embodiments the positive control produces or displays the same or a similar signal that indicates the identity of the specific OP nerve agent and/or OP pesticide. In some embodiments the positive control is enclosed and in other embodiments it is open to the environment. In some embodiments the positive control and/or the negative control is configured on the device body in the same manner as the plurality of spaced apart test spots.

In a further embodiment of the present invention the device can comprise a third fluid reservoir holding a third defined fluid and can be configured to controllably release the third defined fluid to another fluid reservoir and/or the one or more test spots. The third fluid reservoir can be attached to another fluid reservoir and/or the device body. In one embodiment the third defined fluid comprises, consists essentially of, or consists of an oxime and the third fluid reservoir is configured to controllably release the third defined fluid to be in fluid communication with the one or more test spots. In another embodiment the device can further comprise a third reservoir barrier that is attached to the third fluid reservoir and resides between the third fluid reservoir and the test spots. The third reservoir barrier and/or the third fluid reservoir can be configured to controllably release the third defined fluid to the one or more test spots and/or another fluid reservoir. The third fluid reservoir may be located next to, on top of, below, and/or separate from the first fluid reservoir and/or the second fluid reservoir. In some embodiments the third fluid reservoir is detachable from the device body, the first fluid reservoir, the second fluid reservoir, and/or any combination thereof. In some embodiments the third reservoir barrier and/or third fluid reservoir are configured to controllably release the third defined fluid to be in fluid communication with the one or more test spots after the one or more enzymes have had time to react with the substrate in the combined fluid to generate a signal. In this embodiment the oxime will release the substrate, OP nerve agent, and/or OP pesticide from the enzyme. Thus, the third defined fluid will wash the one or more enzymes and allow for the one or more enzymes to be reused for subsequent testing.

In some embodiments of the present invention the device is designed for a single use. In other embodiments of the present invention the device is recyclable for multiple uses.

In some embodiments of the present invention the device is portable. In other embodiments the device is attached at a fixed location. In further embodiments the device is passive, meaning the device requires no external or internal power source. In other embodiments the device requires an external or internal power source. In further embodiments the detection of the signal is accomplished by utilizing another device, such as but not limited to, a microscope, an array detector, a camera, etc. In still further embodiments the detection of the signal is optically detectable without the aid of another device.

In one embodiment the device comprises a set of antidote instructions that describe the treatment and/or decontamination method to utilize depending on the specific OP nerve agent and/or OP pesticide identified.

Another embodiment of the present invention comprises a device for identifying at least one organophosphorus nerve agent, the device comprising:

a device body comprising a plurality of spaced apart test spots, wherein, in operation, the device body is adapted to be in communication with a sample and allow the sample to communicate with each test spot, wherein each test spot comprises at least one enzyme, and wherein different test spots comprise a different enzyme or a different combination of enzymes from the other test spots; and

a first fluid reservoir holding a first defined fluid comprising a substrate, wherein the first fluid reservoir is attached to the device body, and wherein the first fluid reservoir is configured to controllably release the first defined fluid so that the first defined fluid is in fluid communication with the one or more test spots to expose the first defined fluid to the one or more enzymes, whereby the substrate from the first defined fluid reacts with one or more of the enzymes of a respective test spot if an organophosphorus nerve agent is present in the sample to generate a signal that identifies the organophosphorus nerve agent.

A further embodiment of the present invention comprises an active encapsulated mutant of human carboxylesterase 1 in a silicate nanoparticle, wherein the mutant is selected from the group consisting of V146H/L363Q human carboxylesterase 1, L97K human carboxylesterase 1, and V146H/L363E human carboxylesterase 1 and any combination thereof.

The following examples are included to demonstrate various embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### EXAMPLES

### hCE1 Mutant Design

Site-directed mutagenesis was performed with custom primers designed to produce the desired mutations. The GenBank® Database accession number for hCE1 is M73499. Briefly, 100 μM of both sense and anti-sense custom primers were mixed with 50 ng hCE1 cDNA in a pUC9 vector, 200 μM dNTPs, 1×Pfu-BSA buffer, and 2 u Pfu (NEB). Mutations were introduced and amplified through 15 rounds of PCR at 95° C. for 1 minute, 58° C. for 30 seconds, 70° C. for 10 minutes, followed by 1 hour incubation with DPN1 at 37° C. and transformation into chemically competent DH5α cells. Following overnight growth on ampicillian resistant plates, individual colonies were selected, grown overnight in LB medium suspension and the cDNA was isolated with a GeneJET Plasmid Miniprep kit (Fermentas). Mutations were confirmed through DNA sequencing.

Once a successful mutation was identified, the 1.7 kB hCE1 gene was cloned out of pUC9 and ligated into pCIneo for mammalian cell expression. Using custom designed primers containing an EcoR1 restriction site on the sense primer, and a Sma1 restriction site on the anti-sense primer a similar PCR protocol as above was modified to 30 rounds of PCR and a Pfu polymerase extension time of 4 minutes at 70° C. PCR product was then purified with a GeneJET PCR purification kit (Fermentas) and subjected to sequential EcoR1 and Sma1 restriction digests (NEB) for insert preparation. 10 μg pCIneo plasmid was also digested with similar restriction enzymes as well as antartic phosphatase (NEB) prior to ligation. A 1:3 vector to insert ratio was mixed with 1×T4 ligase buffer, 5% PEG 4000, and T4 DNA ligase (NEB) to join the mutated hCE1 insert into pCIneo. The ligation mixture was incubated at room temperature of 1 hour prior to transformation into DH5α cells. Following colony growth on ampicillan resistant plates, individual colonies were grown in LB media, cDNA purified, and proper insertion was confirmed through ethidium bromide-agarose gcl analysis and DNA sequencing.

hCE1 Protein Expression and Purification.

Non-secreted forms wildtype and mutant hCE1 were prepared in COS cells as previously described (Wierdl et al., 2008). To confirm expression, carboxylesterase activity was determined by measuring nanomoles O-nitrophenol produced per minute per milligram protein produced from 3 mM o-nitrophenyl acetate at 420 nM. Mutant carboxylesterase activities were normalized relative to protein expression visualized through western blot analysis. To facilitate measurement of kinetics constants, secreted forms of wildtype and mutant hCE1 were expressed through baculovirus mediated infection of Spodoptera frugiperda Sf9 insect cells and purified as previously described (Morton and Potter, 2000).

Bimolecular Rate Constants of Inhibition with Nerve Agent Analogs.

100 nM of purified mutant enzyme were incubated at room temperature with increasing concentration of stereogenic thiomethylated nerve agent analogs. Aliquots of enzyme inhibited with sarin, soman, and cyclosarin analogs were removed at various time points (up to 1 hour) and the level of remaining enzyme activity was determined by comparing 4-methylumbelliferyl acetate (4-MUA) hydrolysis relative to an uninhibited sample. These data, emission measured at 450 nM following 350 nM excitation, were collected at 37° C. on a Pherastar microplate reader (BMG Labtech) and fit to equation 1 (Aurbek et al., 2006):

$$\frac{Δ t}{Δ ln v} = \frac{{K}_{d}}{{k}_{2}} * \frac{1}{[ IX ] ( 1 - α )} + \frac{1}{{k}_{2}} \qquad ( 1 )$$

where K_{d} was the dissociation constant, k₂ the unimolecular phosphorylation rate constant, the remaining percent enzyme activity, and [IX] the OP analog concentration. α was [S]/(K_{M}+[S]), in which [S] was the substrate concentration and K_{M} was the Michaelis-Menton constant. For these experiments α was 0.91. All experiments were preformed in triplicate and data were analyzed in KaleidaGraph 4 (Synergy Software, Reading, Pa.) to determine kᵢ, where kᵢ=k₂K_{d}.

Spontaneous Reactivation of hCE1 Mutants.

Fifty μL of whole cell COS lysates was inhibited with a ˜1000-fold molar excess of racemic bona fide OP agents sarin, soman, and cyclosarin for 10 minutes. Excess agent was removed by passing inhibited samples over a PD-10 Sephadex G-25 size exclusion column. The column eluate was diluted 10-fold in 0.1 M potassium phosphate buffer, pH 7.4, and tested for level of carboxylesterase activity, or rate of O-nitrophenol formation at 420 nM from 5 mM para-nitrophenyl butyrate (pNPB), relative to an uninhibited sample. Measurements were taken over 60 hours and the rate of reactivation (k_{obs}) and maximal percent recovery (Aₘₐₓ) were determined by fitting the collected data to equation 2:

A=A₀+Aₘₐₓ(1−e^{-k}^{_{abs}}^{t}) (2)

where A was the percent activity at time, t, and A₀ was the initial activity at t=0. The experiments were conducted in triplicate and independently replicated. The data were analyzed in KaleidaGraph 4 software.

All publications, patent applications, patents, accession numbers, sequences and other references cited herein are incorporated by reference in their entireties for the teachings relevant to the sentence and/or paragraph in which the reference is presented.

The foregoing is illustrative of the present invention, and is not to be construed as limiting thereof. The invention is defined by the following claims, with equivalents of the claims to be included therein.

### REFERENCES

Aurbek N, Thiermann H, Szinicz L, Eyer P and Worek F (2006) Analysis of inhibition, reactivation and aging kinetics of highly toxic organophosphorus compounds with human and pig acetylcholinesterase: Toxicology 224(1-2):91-99.
Millard C B, Lockridge 0 and Broomfield C A (1998) Organophosphorus acid anhydride hydrolase activity in human butyrylcholinesterase: synergy results in a somanase. Biochemistry 37(1):237-247.
Morton C L and Potter P M (2000) Comparison of Escherichia coli, Saccharomyces cerevisiae, Pichia pastoris, Spodoptera frugiperda, and COS7 cells for recombinant gene expression. Application to a rabbit liver carboxylesterase. Mol Biotechnol 16(3):193-202.
Wierdl M, Tsurkan L, Hyatt J L, Edwards C C, Hatfield M J, Morton C L, Houghton P J, Danks M K, Redinbo M R and Potter P M (2008) An improved human carboxylesterase for enzyme/prodrug therapy with CPT-11. Cancer Gene Ther 15(3):183-192.

1. A method for identifying an organophosphorus nerve agent and/or an organophosphorus pesticide, comprising:
a) exposing a group of enzymes comprising human carboxylesterase 1, at least one mutant of human carboxylesterase 1, and acetylcholinesterase to a sample, wherein the enzymes are separate from each other and each enzyme binds at least one organophosphorus nerve agent or at least one organophosphorus pesticide;
b) contacting the exposed enzymes with a fluid comprising an oxime and a substrate; and
c) detecting a signal produced upon reaction of the substrate and the exposed enzymes of step b), whereby detection of the signal identifies the organophosphorus nerve agent and/or the organophosphorus pesticide.

2. The method of claim 1, wherein the organophosphorus pesticide is paraoxon.

3. A method for identifying an organophosphorus nerve agent, comprising:
a) exposing a group of enzymes comprising acetylcholinesterase and at least one mutant of human carboxylesterase 1 to a sample, wherein the enzymes are separate from each other and each enzyme binds at least one organophosphorus nerve agent;
b) contacting the exposed enzymes with a fluid comprising an oxime and a substrate; and
c) detecting a signal produced upon reaction of the substrate and the exposed enzymes of step b), whereby detection of the signal identifies the organophosphorus nerve agent.

4. The method of claim 1, wherein the organophosphorus nerve agent is selected from the group consisting of sarin, soman, cyclosarin, tabun, VX, VR, and any combination thereof.

5. A method for identifying an organophosphorus nerve agent, comprising:
a) exposing an enzyme to a sample, wherein the enzyme is a mutant of human carboxylesterase 1 and the enzyme binds at least one organophosphorus nerve agent;
b) contacting the exposed enzyme with a fluid comprising an oxime and a substrate; and
c) detecting a signal produced upon reaction of the substrate and the exposed enzyme of step b), whereby detection of the signal identifies the organophosphorus nerve agent.

6. The method of claim 5, wherein the organophosphorus nerve agent is selected from the group consisting of sarin, soman, cyclosarin, VX, VR, and any combination thereof.

7. The method of claim 1, wherein the mutant of human carboxylesterase 1 is selected from the group consisting of V146H/L363Q human carboxylesterase 1, L97K human carboxylesterase 1, V146H/L363E human carboxylesterase 1, and any combination thereof.

8. A method for identifying an organophosphorus nerve agent, comprising:
a) exposing an enzyme selected from the group consisting of acetylcholinesterase, V146H/L363Q human carboxylesterase 1, L97K human carboxylesterase 1, V146H/L363E human carboxylesterase 1, and any combination thereof to a sample, wherein each enzyme binds at least one organophosphorus nerve agent selected from the group consisting of sarin, soman, cyclosarin, tabun, VX, VR, and any combination thereof;
b) contacting the exposed enzyme with a fluid comprising an oxime and a substrate; and
c) detecting a signal produced upon reaction of the substrate and the exposed enzyme of step b), whereby detection of the signal identifies the organophosphorus nerve agent.

9. A method for identifying an organophosphorus nerve agent and/or an organophosphorus pesticide, comprising:
a) exposing an enzyme selected from the group consisting of acetylcholinesterase, human carboxylesterase 1, V146H/L363Q human carboxylesterase 1, L97K human carboxylesterase 1, V146H/L363E human carboxylesterase 1, and any combination thereof to a sample, wherein each enzyme binds at least one organophosphorus nerve agent selected from the group consisting of sarin, soman, cyclosarin, tabun, VX, VR, and any combination thereof or the organophosphorus pesticide paraoxon;
b) contacting the exposed enzyme with a fluid comprising an oxime and a substrate; and
c) detecting a signal produced upon reaction of the substrate and the exposed enzyme of step b), whereby detection of the signal identifies the organophosphorus nerve agent and/or the organophosphorus pesticide.

10. The method of claim 1, wherein the oxime is diacetyl monoxime, obidoxime or a combination thereof.

11. The method of claim 1, wherein the substrate is a colorimetric substrate.

12. The method of claim 1, wherein the substrate is p-nitrophenyl butyrate or o-nitrophenyl acetate.

13. The method of claim 1, wherein the method further comprises rinsing the exposed enzyme with a second fluid comprising an oxime.

14. The method of claim 1, wherein the enzyme is encapsulated in a silicate nanoparticle.

15. The method of claim 1, wherein the detection of the signal is passive.

16. The method of claim 1, wherein the signal is optically detectable.

17. The method of claim 1, wherein the signal is electrically detectable.

18. The method of claim 1, wherein the organophosphorus nerve agent and/or organophosphorus pesticide has a concentration from about 1 μM to about 10 mM.

19. The method of claim 1, wherein the identification of the organophosphorus nerve agent and/or organophosphorus pesticide is accomplished in about 30 seconds to about 1 hour.

20. A device for identifying at least one organophosphorus nerve agent and/or at least one organophosphorus pesticide, the device comprising:
a device body comprising a plurality of spaced apart test spots, wherein, in operation, the device body is adapted to be in communication with a sample and allow the sample to communicate with each test spot, wherein each test spot comprises at least one enzyme, and wherein different test spots comprise a different enzyme or a different combination of enzymes from the other test spots; and
a first fluid reservoir holding a first defined fluid comprising a substrate, wherein the first fluid reservoir is attached to the device body, and wherein the first fluid reservoir is configured to controllably release the first defined fluid so that the first defined fluid is in fluid communication with the one or more test spots to expose the first defined fluid to the one or more enzymes, whereby the substrate from the first defined fluid reacts with one or more of the enzymes of a respective test spot if an organophosphorus nerve agent and/or organophosphorus pesticide is present in the sample to generate a signal that identifies the organophosphorus nerve agent and/or organophosphorus pesticide.

21. The device of claim 20, wherein the first defined fluid further comprises an oxime.

22. The device of claim 20, wherein the device further comprises a first reservoir barrier that resides between the first fluid reservoir and the test spots and is configured to controllably release the first defined fluid.

23. The device of claim 20, wherein the device further comprises a second fluid reservoir holding a second defined fluid comprising an oxime, wherein the second fluid reservoir is attached to the first fluid reservoir and the second fluid reservoir is configured to controllably release the second defined fluid.

24. The device of claim 23, wherein the device further comprises a second reservoir barrier that resides between the first fluid reservoir and the second fluid reservoir and is configured to controllably release the second defined fluid.

25. The device of claim 23, wherein the second defined fluid is controllably released into the first defined fluid to produce a combined fluid, and wherein the first fluid reservoir or the first reservoir barrier is configured to controllably release the combined fluid.

26. The device of claim 23, wherein the second defined fluid is controllably released to be in fluid communication with the one or more test spots.

27. A device for identifying at least one organophosphorus nerve agent and/or at least one organophosphorus pesticide, the device comprising:
a device body comprising a plurality of spaced apart test spots, wherein, in operation, the device body is adapted to be in communication with a sample and allow the sample to communicate with each test spot, wherein each test spot comprises at least one enzyme, and wherein different test spots comprise a different enzyme or a different combination of enzymes from the other test spots;
a first fluid reservoir holding a first defined fluid comprising a substrate, wherein the first fluid reservoir is attached to the device body; and
a second fluid reservoir holding a second defined fluid comprising an oxime, wherein the second fluid reservoir is attached to the first fluid reservoir so that the first defined fluid is separated from the second defined fluid, and wherein the second fluid reservoir is configured to controllably release the second defined fluid into the first defined fluid to produce a combined fluid and the first fluid reservoir is configured to controllably release the combined fluid so that the combined fluid is in fluid communication with the one or more test spots to expose the combined fluid to the one or more enzymes whereby the substrate from the first defined fluid reacts with one or more of the enzymes of a respective test spot if an organophosphorus nerve agent and/or organophosphorus pesticide is present in the sample to generate a signal that identifies the organophosphorus nerve agent and/or organophosphorus pesticide.

28. The device of claim 27, wherein the device further comprises a first reservoir barrier that resides between the first fluid reservoir and the test spots and is configured to controllably release the combined fluid.

29. The device of claim 27, wherein the device further comprises a second reservoir barrier that resides between the first fluid reservoir and the second fluid reservoir and is configured to controllably release the second defined fluid into the first defined fluid to produce a combined fluid.

30. The device of claim 27, wherein each test spot comprises an enzyme selected from the group consisting of acetylcholinesterase, mutant acetylcholinesterases, human carboxycholinesterase, mutant carboxylesterases, and any combination thereof.

31. The device of claim 30, wherein the mutant carboxylesterases are selected from the group consisting of V146H/L363Q human carboxylesterase 1, L97K human carboxylesterase 1, V146H/L363E human carboxylesterase 1, and any combination thereof.

32. The device of claim 27, wherein the organophosphorus nerve agent is selected from the group consisting of sarin, soman, cyclosarin, tabun, VX, VR, and any combination thereof.

33. The device of claim 27, wherein organophosphorus pesticide is paraoxon.

34. The device of claim 27, wherein the oxime is diacetyl monoxime, obidoxime, or a combination thereof.

35. The device of claim 27, wherein the substrate is a colorimetric substrate.

36. The device of claim 27, wherein the substrate is p-nitrophenyl butyrate or o-nitrophenyl acetate.

37. The device of claim 27, wherein the device further comprises a third fluid reservoir holding a third defined fluid comprising an oxime, wherein the third fluid reservoir is attached to the device body and is configured to controllably release the third defined fluid to be in fluid communication with the one or more test spots.

38. The device of claim 37, wherein the device further comprises a third reservoir barrier that is attached to the third fluid reservoir and is configured to controllably release the third defined fluid to be in fluid communication the one or more test spots.

39. The device of claim 27, wherein the device detects and identifies the organophosphorus nerve agent and/or organophosphorus pesticide in a concentration range from about 1 μM to about 10 mM.

40. The device of claim 27, wherein the device detects and identifies the organophosphorus nerve agent and/or organophosphorus pesticide in about 30 seconds to about 1 hour.

41. The device of claim 27, wherein the at least one enzyme is encapsulated in a silicate nanoparticle.

42. The device of claim 27, wherein the signal is an optically detectable signal.

43. The device of claim 27, wherein the signal is an electrically detectable signal.

44. The device of claim 27, wherein the one or more enzymes react with the substrate in the first defined fluid, if an organophosphorus nerve agent and/or organophosphorus pesticide is present in the sample, to passively generate a signal.

45. An active encapsulated mutant of human carboxylesterase 1 in a silicate nanoparticle, wherein the mutant is selected from the group consisting of V146H/L363Q human carboxylesterase 1, L97K human carboxylesterase 1, and V146H/L363E human carboxylesterase 1 and any combination thereof.
